(19) United States
(12) Patent Application Publication
(10) Pub. No.: US 2025/0277072 A1
(43) Pub. Date: Sep. 4, 2025

(54) CURABLE COMPOSITION, CURED PRODUCT, OPTICAL MATERIAL, DIFFRACTIVE OPTICAL ELEMENT, AND MONOFUNCTIONAL (METH)ACRYLIC ACID THIOESTER MONOMER

(71) Applicant: FUJIFILM Corporation, Tokyo (JP)

(72) Inventors: Naozumi SHIRAIWA, Kanagawa (JP); Naoyuki MOROOKA, Kanagawa (JP)

(73) Assignee: FUJIFILM Corporation, Tokyo (JP)

(21) Appl. No.: 19/214,013

(22) Filed: May 20, 2025

Related U.S. Application Data
(63) Continuation of application No. PCT/JP2023/044705, filed on Dec. 13, 2023.

(30) Foreign Application Priority Data
Dec. 16, 2022 (JP) 2022-201313

(51) Int. Cl. C08F 220/38; C07C 327/22; C07C 327/28; G02B 1/04; G02B 5/18

(52) U.S. Cl. CPC C08F 220/382; C07C 327/22; C07C 327/28; G02B 1/04; G02B 5/1814

(57) ABSTRACT

The present invention provides a curable composition including a monofunctional (meth)acrylic acid thioester monomer represented by General Formula (1) and a difunctional (meth)acrylic acid thioester monomer; a cured product, an optical material, and a diffractive optical element that use the curable composition; a monofunctional (meth)acrylic acid thioester monomer represented by General Formula (1); and a curable composition including the monofunctional (meth)acrylic acid thioester monomer represented by General Formula (1).

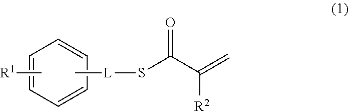

In the formula, R¹ represents a group containing an arylene group and an S atom, and L represents a single bond or a methylene group. R² represents a hydrogen atom or a methyl group.

### CROSS-REFERENCE TO RELATED APPLICATIONS

This application is a Continuation of PCT International Application No. PCT/JP2023/044705 filed on Dec. 13, 2023, which claims priority under 35 U.S.C. § 119 (a) to Japanese Patent Application No. 2022-201313 filed in Japan on Dec. 16, 2022. Each of the above applications is hereby expressly incorporated by reference, in its entirety, into the present application.

### BACKGROUND OF THE INVENTION

### 1. Field of the Invention

The present invention relates to a curable composition, a cured product, an optical material, a diffractive optical element, and a monofunctional (meth) acrylic acid thioester monomer.

### 2. Description of the Related Art

In recent years, there have been active studies on optical resins that are expected to be applied to optical members such as diffractive optical elements, which require a high refractive index, and progress has also been made in the development of monomers that exhibit a high refractive index as raw materials for optical resins.

For example, JP2020-37693A describes a polymerizable compound having a specific structure, which has 1 to 3 arylthio groups and 1 polymerizable group on a benzene ring, as a polymerizable compound having a refractive index np of 1.650 or more at 25° C.

### SUMMARY OF THE INVENTION

A nanofabrication technology called an imprinting technology, in which a mold having a microstructure with a variety of patterns having a size of nanometers to several hundred micrometers is embossed into a material such as a resin to transfer the patterns to a surface of the material, is known.

In order to transfer fine patterns having a size of nanometers to several hundred micrometers formed on a mold to a material such as a resin to be imprinted with high accuracy, it is required that the material does not contain inorganic particles and has low viscosity.

The imprinting consists of a step of applying a material onto a substrate, a step of pressing a mold in a state of being embossed, a step of fixing a transferred pattern by photocuring or thermal curing, and a step of demolding. The material used for imprinting is temporarily stored in a tank before use as in a case of an ink in ink jetting, before proceeding to the above-mentioned imprinting step, so it is required that crystals do not precipitate and turbidity does not occur in the storage tank (hereinafter, also referred to as “temporal stability”).

In addition, a compound having an aromatic ring in a molecular structure thereof is expected to have a high refractive index, but often exhibits high viscosity. Therefore, from the viewpoint of application to imprinting, it is required to achieve both a high refractive index and low viscosity at a high level.

Furthermore, from the viewpoint of manufacturing an optical material having a desired microstructure pattern with high accuracy by an imprinting technology, it is also important that resin chipping does not occur in a case of being released from a mold.

As a result of studies by the present inventors, it has been found that, in a case where the polymerizable compound described in JP2020-37693A is used in a curable composition containing a polymerizable compound, a polymerization initiator, a monomer for dilution, and the like in order to obtain a cured product having a high refractive index, resin chipping is likely to occur in a case of being released from a mold, crystal precipitation and/or turbidity is likely to occur in the composition, and it cannot be said that both a high refractive index of the cured product and a low viscosity of the composition are sufficiently achieved.

An object of the present invention is to provide a curable composition which makes it possible to obtain a cured product which is less susceptible to resin chipping in a case of being released from a mold, which exhibits excellent temporal stability in a state of a curable composition before a curing reaction, and which also makes it possible to achieve a low viscosity of the composition and an increase in refractive index of the obtained cured product. In addition, another object of the present invention is to provide a cured product obtained from the curable composition, an optical material and a diffractive optical element each including the cured product, and a monomer contained in the curable composition.

The above-mentioned objects of the present invention have been achieved by the following means.

<1>

A curable composition comprising: a monofunctional (meth)acrylic acid thioester monomer represented by General Formula (1); and a difunctional (meth)acrylic acid thioester monomer.

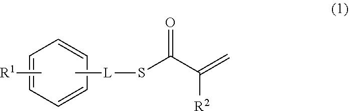

In the formula, R¹ represents a group containing an arylene group and an S atom, and L represents a single bond or a methylene group. R² represents a hydrogen atom or a methyl group.

<2>

The curable composition according to <1>, in which a refractive index nD of a liquid at 25° C. is 1.650 or more.

<3>

The curable composition according to <1> or <2>, in which the monofunctional (meth)acrylic acid thioester monomer represented by General Formula (1) includes a monofunctional (meth)acrylic acid thioester monomer represented by General Formula (2).

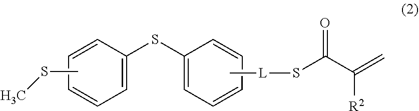

In the formula, L and R² have the same definition as L and R² described above.

<4>

The curable composition according to any one of <1> to <3>, in which the monofunctional (meth)acrylic acid thioester monomer represented by General Formula (1) includes a monomer in which L in General Formula (1) is a single bond.

<5>

The curable composition according to <3>, in which the monofunctional (meth) acrylic acid thioester monomer represented by General Formula (1) includes a monomer in which at least one of a bonding position of —S—CH₃ on a benzene ring or a bonding position of L on a benzene ring in General Formula (2) is a meta position and the L is a single bond.

<6>

The curable composition according to <5>, in which the monofunctional (meth)acrylic acid thioester monomer represented by General Formula (1) includes a monofunctional (meth)acrylic acid thioester monomer represented by General Formula (3).

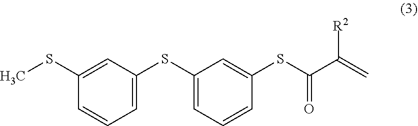

In the formula, R² has the same definition as R² described above.

<7>

The curable composition according to any one of <1> to <6>, in which the curable composition is for imprinting.

<8>

A cured product of the curable composition according to any one of <1> to <7>.

<9>

An optical material comprising: the cured product according to <8>.

<10>

A diffractive optical element which is formed of the cured product according to <8>, comprising: a surface having a diffraction grating shape.

<11>

A monofunctional (meth)acrylic acid thioester monomer represented by General Formula (1).

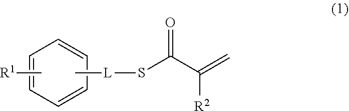

In the formula, R¹ represents a group containing an arylene group and an S atom, and L represents a single bond or a methylene group. R² represents a hydrogen atom or a methyl group.

<12>

A curable composition comprising: the monofunctional (meth)acrylic acid thioester monomer represented by General Formula (1) according to <11>.

In the present invention, in a case where there are a plurality of substituents, linking groups, or the like (hereinafter, referred to as substituents or the like) represented by a specific reference numeral or formula, or in a case where a plurality of substituents or the like are defined simultaneously, unless otherwise specified, the substituents or the like may be the same as or different from each other (regardless of the presence or absence of the expression “each independently”, the substituents or the like may be the same as or different from each other). The same applies to the definition of the number of substituents or the like. In a case where a plurality of substituents or the like come close to each other (particularly in a case where a plurality of substituents or the like are adjacent to each other), the substituents or the like may be linked to each other to form a ring, unless otherwise specified. In addition, unless otherwise specified, a ring, for example, an alicyclic ring, an aromatic ring, or a heterocyclic ring may be further condensed to form a fused ring.

In the present invention, unless otherwise specified, in a case where E and Z configurations for double bond are present in a molecule, the double bond may be any one thereof or may be a mixture thereof.

In addition, in the present invention, unless otherwise specified, in a case where a compound has one or two or more asymmetric carbons, for the stereochemistry of such asymmetric carbons, either an (R)-form or an(S)-form can be independently taken. As a result, the compound may be a mixture of stereoisomers such as optical isomers or diastereoisomers, or may be racemic.

In addition, in the present invention, the expression of the compound and the monomer is meant to include a compound or monomer in which the structure is partially modified within a range where the effect of the present invention is not impaired. Furthermore, the compound and the monomer that are not specifically described as substituted or unsubstituted are intended to mean that the compound or the monomer may have any substituent within a range where the effect of the present invention is not impaired. For example, in the monomer represented by General Formula (1), the benzene ring having R¹ and -L-S—C(═O)C(R²)═CH₂ may be unsubstituted or may have any substituent. Examples of any substituent include a saturated or unsaturated aliphatic hydrocarbon group which may be chain-like, branched, or cyclic, an aromatic hydrocarbon group, an aromatic heterocyclic group, an alkoxy group, an alkylthio group, an aryloxy group, an arylthio group, and a halogen atom. From the viewpoint of imprinting applications, it is preferable that the benzene ring having R¹ and -L-S—C(═O)C(R²)═CH₂ is unsubstituted, or in a case where the benzene ring having R¹ and -L-S—C(═O)C(R²)—CH₂ has a substituent, the substituent is a halogen atom.

In the present invention, with respect to a substituent that is not specified as substituted or unsubstituted (the same applies to a linking group and a ring), this means that the group may have any substituent within a range that does not impair the desired effect. For example, the term “alkyl group” means to include both an unsubstituted alkyl group and a substituted alkyl group.

In the present invention, in a case where the number of carbon atoms in a certain group is defined, the number of carbon atoms refers to the number of carbon atoms in the entire group unless otherwise specified in the present invention or the present specification. In other words, in a case where this group is in a form in which it further has a substituent, the number of carbon atoms refers to the total number of carbon atoms including the substituent.

In the present invention, any numerical range represented by using “to” refers to a range including numerical values described before and after “to” as a lower limit value and an upper limit value, respectively.

In the curable composition according to the embodiment of the present invention, each component may be used alone or in combination of two or more thereof. The same applies to a cured product, an optical material, and a diffractive optical element, which are obtained from the curable composition according to the embodiment of the present invention.

In the present invention, “(meth)acrylate” refers to either or both of acrylate and methacrylate, “(meth)acrylic acid” refers to either or both of acrylic acid and methacrylic acid and “(meth)acryloyl” refers to either or both of acryloyl and methacryloyl. In the present invention, a monomer is distinguished from an oligomer and a polymer based on the molecular weight, and a compound having a weight-average molecular weight of 1000 or less is referred to as the monomer.

In the present invention, the alkyl group refers to a linear or branched alkyl group.

The number of carbon atoms in the alkyl group is preferably 1 to 20, more preferably 1 to 10, still more preferably 1 to 7, and particularly preferably 1 to 5.

Examples of the alkyl group include a methyl group, an ethyl group, a propyl group, an isopropyl group, a butyl group, an isobutyl group, a sec-butyl group, a tert-butyl group, a pentyl group, a 1-methylbutyl group, a 3-methylbutyl group, a hexyl group, a 1-methylpentyl group, a 4-methylpentyl group, a heptyl group, a 1-methylhexyl group, a 5-methylhexyl group, a 2-ethylhexyl group, an octyl group, a 1-methylheptyl group, a nonyl group, a 1-methyloctyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group, among which a methyl group or an ethyl group is preferable.

The same applies to the alkyl group in a group containing an alkyl group (an alkoxy group, an alkylsulfanyl group, an alkoxycarbonyl group, an acyl group, an acyloxy group, or the like).

In addition, the alkyl group may have a substituent. Examples of such an alkyl group having a substituent include a halogenated alkyl group and a hydroxyalkyl group.

In the present invention, the alkylene group may be, for example, a group obtained by removing one hydrogen atom bonded to a carbon atom in the above-mentioned alkyl group. The alkylene group may be a linear alkylene group or a branched alkylene group. Examples of the alkylene group include an ethylene group, a propylene group, and a butylene group.

In the present invention, the monovalent aromatic hydrocarbon group refers to a monovalent group obtained by removing any one hydrogen atom from an aromatic hydrocarbon ring which may be a monocyclic ring or a fused ring. The monovalent aromatic hydrocarbon group is preferably an aromatic hydrocarbon group having 6 to 14 carbon atoms, and examples thereof include a phenyl group, an 1-naphthyl group, a 2-naphthyl group, an 1-anthracenyl group, a 2-anthracenyl group, a 9-anthracenyl group, an 1-phenanthryl group, a 2-phenanthryl group, a 3-phenanthryl group, a 4-phenanthryl group, and a 9-phenanthryl group. Among these groups, a phenyl group is preferable.

In the present invention, the divalent aromatic hydrocarbon group (arylene group) refers to a divalent group obtained by removing any one hydrogen atom from the above-mentioned monovalent aromatic hydrocarbon group. Examples of the divalent aromatic hydrocarbon group include a phenylene group, a naphthylene group, and a phenanthrylene group, with a phenylene group being preferred, and a 1,3-phenylene group or a 1,4-phenylene group being more preferred.

In the present invention, examples of the halogen atom include a fluorine atom, a chlorine atom, a bromine atom, and an iodine atom.

The curable composition according to the embodiment of the present invention exhibits excellent temporal stability, makes it possible to obtain a cured product which is less susceptible to resin chipping in a case of being released from a mold, and makes it possible to achieve both a low viscosity of the curable composition and a high refractive index of the obtained cured product at an excellent level. The cured product, optical material, and diffractive optical element according to the embodiment of the present invention can exhibit a high refractive index. The monofunctional (meth)acrylic acid thioester monomer according to the embodiment of the present invention makes it possible to obtain the above-mentioned curable composition according to the embodiment of the present invention, which exhibits the excellent characteristics.

### BRIEF DESCRIPTION OF THE DRAWINGS

FIG. 1 is a schematic cross-sectional view schematically showing a method of manufacturing a cured product according to the embodiment of the present invention by an imprinting technology using a curable composition according to the embodiment of the present invention.

### DESCRIPTION OF THE PREFERRED EMBODIMENTS

### Curable Composition

The curable composition according to the embodiment of the present invention contains a monofunctional (meth)acrylic acid thioester monomer represented by General Formula (1) and a difunctional (meth)acrylic acid thioester monomer.

The curable composition according to the embodiment of the present invention means a composition which has curing properties and which makes it possible to obtain a cured product (resin) by a curing reaction.

The monofunctional (meth)acrylic acid thioester monomer contained in the curable composition according to the embodiment of the present invention is a compound in which a (meth)acryloylthio group and a benzene ring are linked by a single bond or a methylene group represented by L, and which has, on the benzene ring, a group containing an arylene group and an S atom, represented by R¹, as a substituent, as represented by General Formula (1) which will be described later.

The monofunctional (meth)acrylic acid thioester monomer represented by General Formula (1) has the above-mentioned specific chemical structure, and thus, in a case of being made into a curable composition, the monofunctional (meth)acrylic acid thioester monomer exhibits excellent temporal stability in a state of a curable composition before a curing reaction, makes it possible to obtain a cured product in which the suppression of resin chipping in a case of being released from a mold is achieved, and furthermore, makes it possible to achieve both a low viscosity of the curable composition and a high refractive index of the obtained cured product at a high level. In addition, the curable composition according to the embodiment of the present invention, which contains the monofunctional (meth)acrylic acid thioester monomer represented by General Formula (1) and the difunctional (meth)acrylic acid thioester monomer, makes it possible to obtain a cured product in which the occurrence of resin chipping in a case of being released from a mold can be sufficiently suppressed to a low level.

Hereinafter, the components contained in the curable composition according to the embodiment of the present invention will be described in order.

### <Monofunctional (Meth)Acrylic Acid Thioester Monomer Represented by General Formula (1)>

The curable composition according to the embodiment of the present invention contains a monofunctional (meth)acrylic acid thioester monomer represented by General Formula (1) (hereinafter, also simply referred to as “monomer represented by General Formula (1)”).

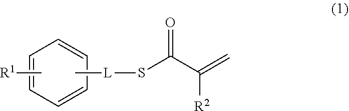

In the formula, R¹ represents a group containing an arylene group and an S atom, and L represents a single bond or a methylene group. R² represents a hydrogen atom or a methyl group.

R¹ represents a group containing an arylene group and an S atom.

In the present invention, the “group containing an arylene group and an S atom” means that R¹ is a group containing an arylene group and an S atom, and other structures are not particularly limited as long as the effects of the present invention are exhibited.

The S atom contained in R¹ is contained as a thioether bond represented by —S—. In this regard, R¹ does not have a structure in which two or more S atoms are linked (a disulfide structure or a polysulfide structure in which three or more S atoms are linked).

The number of S atoms contained in R¹ may be 1 or more and is, for example, preferably 1 to 4, more preferably 2 to 4, still more preferably 2 or 3, and particularly preferably 2.

Examples of the arylene group contained in R¹ include a phenylene group consisting of a monocyclic aromatic hydrocarbon ring and an arylene group consisting of an aromatic hydrocarbon ring formed by condensing two or more rings, among which a phenylene group is preferable.

The number of arylene groups contained in R¹ may be one or more and is, for example, preferably 1 or 2 and more preferably 1.

The arylene group contained in R¹ may be unsubstituted or may have a substituent within a range where the effect of the present invention is not impaired. Examples of the substituent which may be contained in the arylene group contained in R¹ include a halogen atom, a sulfanyl group, and an alkylsulfanyl group, among which a halogen atom is preferable.

In a case where the arylene group contained in R¹ has a substituent, it means that the arylene group has a substituent at a position other than two bonding sites. For example, in a case where the 1,3-phenylene group contained in R¹ has a substituent, it means that the phenylene group has a substituent at any position of 2, 4, 5, or 6 other than two bonding sites.

The number of carbon atoms in the alkylsulfanyl group which may be contained in the arylene group contained in R¹ is preferably 1 to 6, more preferably 1 to 4, still more preferably 1 or 2, and particularly preferably 1.

In a case where the arylene group contained in R¹ has a substituent, the number of substituents is not particularly limited and can be set to, for example, 1 to 4, and is preferably 1 to 3, more preferably 1 or 2, and still more preferably 1.

Above all, the arylene group contained in R¹ is preferably unsubstituted or has a halogen atom as a substituent, and is more preferably unsubstituted.

R¹ may contain a structure other than the above-mentioned arylene group and S atom, and preferably contains, for example, an alkylene structure.

The number of carbon atoms in the alkylene group which can be contained in R¹ is preferably 1 to 6, more preferably 1 to 4, still more preferably 1 or 2, and particularly preferably 1.

The alkylene structure which can be contained in R¹ may be unsubstituted or may have a substituent within a range where the effect of the present invention is not impaired. Examples of the substituent which may be contained in the alkylene structure which can be contained in R¹ include a halogen atom, a sulfanyl group, and an alkylsulfanyl group, among which a halogen atom is preferable.

In a case where the alkylene group which can be contained in R¹ has a substituent, it means that the alkylene group has a substituent at a position other than two bonding sites.

As the alkylsulfanyl group which may be contained in the alkylene structure which can be contained in R¹, the description of the alkylsulfanyl group which may be contained in the arylene group contained in R¹ described above can be preferably applied.

Above all, the alkylene structure which can be contained in R¹ is preferably unsubstituted or has a halogen atom as a substituent, and is more preferably unsubstituted.

Since R¹ is a monovalent group, it contains an arylene group and an S atom, and has an H atom on the other side of the arylene group, —S— or alkylene group, which is a terminal portion of R¹. For example, in a case of being described using the monomer A-1 represented by General Formula (1) in Examples which will be given later, R¹ is a group having an H atom at methylene in —S-phenylene-S-methylene-.

The total number of atoms other than hydrogen atoms constituting R¹ is preferably 7 to 20, more preferably 9 to 18, still more preferably 9 to 15, particularly preferably 9 to 11, and most preferably 9.

R¹ is preferably a group represented by —S-arylene-S-alkylene-H or a group represented by —S-arylene-S-arylene-S-alkylene-H, more preferably a group represented by —S-arylene-S-alkylene-H, still more preferably a group represented by —S-phenylene-S-alkylene-H, and particularly preferably a group represented by —S-phenylene-S-methylene-H.

L represents a single bond or a methylene group. The methylene group means a group represented by >CH₂.

L is preferably a single bond from the viewpoint of further increasing the refractive index nD of the liquid of the curable composition according to the embodiment of the present invention and the refractive index nD of the cured product obtained by curing the curable composition according to the embodiment of the present invention.

Therefore, the monomer represented by General Formula (1) preferably includes a monomer in which L in General Formula (1) is a single bond.

In the monomer represented by General Formula (1), the content of the monomer in which L in General Formula (1) is a single bond is preferably 35% by mass or more, more preferably 50% by mass or more, still more preferably 65% by mass or more, and particularly preferably 80% by mass or more, and may be 100% by mass.

R² represents a hydrogen atom or a methyl group.

R² is preferably a hydrogen atom from the viewpoint of further increasing the refractive index nD of the liquid of the curable composition according to the embodiment of the present invention and the refractive index nD of the cured product obtained by curing the curable composition according to the embodiment of the present invention.

Therefore, the monomer represented by General Formula (1) preferably includes a monomer in which R² in General Formula (1) is a hydrogen atom.

In the monomer represented by General Formula (1), the content of the monomer in which R² in General Formula (1) is a hydrogen atom is preferably 35% by mass or more, more preferably 50% by mass or more, still more preferably 65% by mass or more, and particularly preferably 80% by mass or more, and may be 100% by mass.

In General Formula (1), with regard to a relationship between the bonding positions of R¹ and L on a benzene ring, L may be bonded to any of an ortho position, a meta position, and a para position with respect to a bonding position of R¹, and is preferably bonded to a meta position.

From the viewpoint of further increasing the refractive index nD of the liquid of the curable composition according to the embodiment of the present invention and the refractive index nD of the cured product obtained by curing the curable composition according to the embodiment of the present invention, the monomer represented by General Formula (1) preferably includes a monofunctional (meth)acrylic acid thioester monomer represented by General Formula (2) (hereinafter, also simply referred to as “monomer represented by General Formula (2)”).

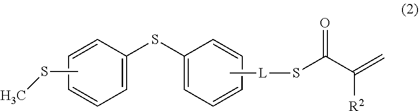

In the formula, L and R² have the same definition as L and R² in General Formula (1).

That is, L is preferably a single bond and R² is preferably a hydrogen atom.

The content of the monofunctional (meth)acrylic acid thioester monomer represented by General Formula (2) in the monomer represented by General Formula (1) is preferably 35% by mass or more, more preferably 50% by mass or more, still more preferably 65% by mass or more, and particularly preferably 80% by mass or more, and may be 100% by mass.

The bonding position of L on the benzene ring with respect to the bonding position of —S-phenylene-S—CH₃ on the benzene ring located on the right side in General Formula (2) may be any of an ortho position, a meta position, and a para position, and is preferably a meta position.

In addition, the bonding position of —S—CH₃ on the benzene ring with respect to the bonding position of —S-phenylene-L-S—C(═O)C(R²)═CH₂ on the benzene ring located on the left side in General Formula (2) may be any of an ortho position, a meta position, and a para position, and is preferably a meta position.

From the viewpoint of further increasing the temporal stability of the composition and further decreasing the viscosity of the composition, it is preferable that at least one of the bonding position of —S—CH₃ on the benzene ring or the bonding position of L on the benzene ring in General Formula (2) is a meta position and L is a single bond, and from the viewpoint of further decreasing the viscosity of the composition, it is more preferable that both of the bonding positions in General Formula (2) are meta positions and L is a single bond.

In the present invention, “the bonding position of L on the benzene ring” means the bonding position of L on the benzene ring with respect to the bonding position of —S-phenylene-S—CH₃ on the benzene ring located on the right side in General Formula (2), and “the bonding position of —S—CH₃ on the benzene ring” means the bonding position of —S—CH₃ on the benzene ring with respect to the bonding position of —S-phenylene-L-S—C(═O)C(R²)═CH₂ on the benzene ring located on the left side in General Formula (2).

Therefore, from the viewpoint of further increasing the temporal stability of the composition and further decreasing the viscosity of the composition, it is preferable that the monomer represented by General Formula (1) includes a monomer in which at least one of the bonding position of —S—CH₃ on the benzene ring or the bonding position of L on the benzene ring in General Formula (2) is a meta position and L is a single bond, and from the viewpoint of further decreasing the viscosity of the composition, it is more preferable that the monomer represented by General Formula (1) includes a monofunctional (meth)acrylic acid thioester monomer represented by General Formula (3) (hereinafter, also simply referred to as “monomer represented by General Formula (3)”).

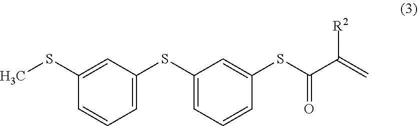

In the formula, R² has the same definition as R² in General Formula (1).

That is, R² is preferably a hydrogen atom.

In the monomer represented by General Formula (1), the content of the monomer in which at least one of the bonding position of —S—CH₃ on the benzene ring or the bonding position of L on the benzene ring in General Formula (2) is a meta position, and L is a single bond is preferably 35% by mass or more, more preferably 50% by mass or more, still more preferably 65% by mass or more, and particularly preferably 80% by mass or more, and may be 100% by mass.

The above description of “the content of the monomer in which at least one of the bonding position of —S—CH³ on the benzene ring or the bonding position of L on the benzene ring in General Formula (2) is a meta position, and L is a single bond” can also be applied to “the content of the monofunctional (meth)acrylic acid thioester monomer represented by General Formula (3)” in the monomer represented by General Formula (1).

Hereinafter, preferred specific examples of the monomer represented by General Formula (1) are listed, but the present invention is not limited to these monomer compounds.

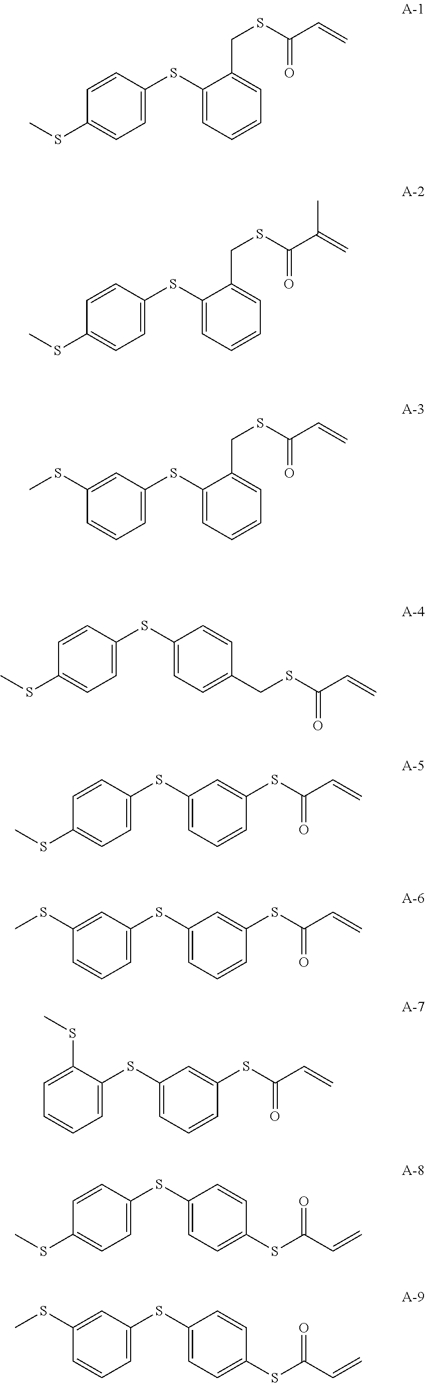

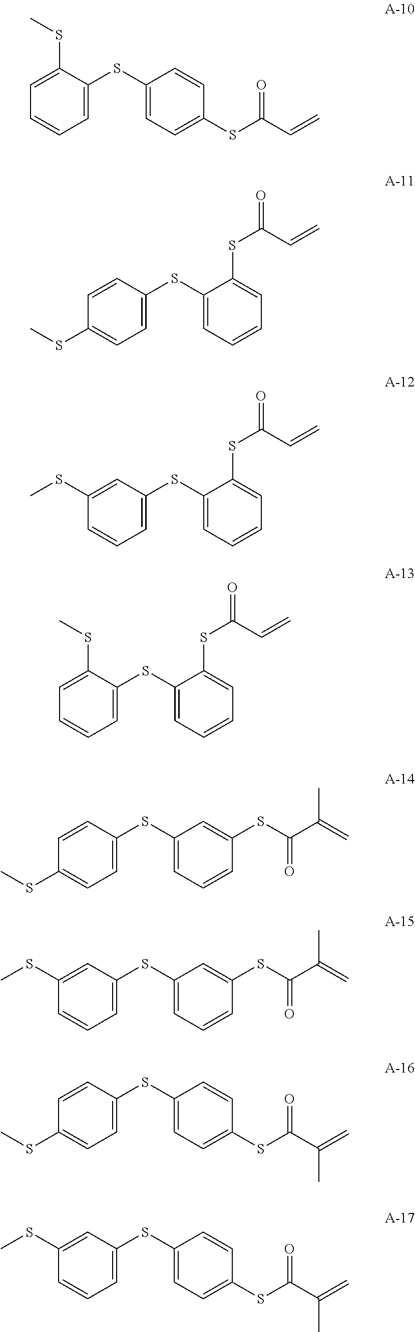

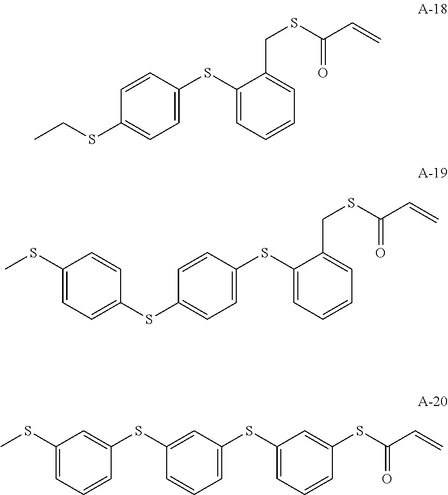

The molecular weight of the monomer represented by General Formula (1) is preferably 270 to 600, more preferably 300 to 600, and still more preferably 300 to 500.

The monomer represented by General Formula (1) can be synthesized by a common method. For example, the monomer represented by General Formula (1) can be synthesized with reference to the synthesis method described in Org. Lett., 2004, Vol. 6, No. 24, pp. 4587 to 4590, the synthesis method described in US2003/0195270A, and the like. In addition, the monomer represented by General Formula (1) can be synthesized as appropriate with reference to the methods described in Examples, and the like.

The content of the monomer represented by General Formula (1) in the curable composition according to the embodiment of the present invention can be set to, for example, 60% by mass or more, and from the viewpoint of further improving (further increasing) the refractive index nD of the liquid of the curable composition according to the embodiment of the present invention and the refractive index nD of the cured product obtained by curing the curable composition according to the embodiment of the present invention (both of which are the refractive indexes at 25° C. and a wavelength of 589 nm), the content of the monomer represented by General Formula (1) is preferably 65% by mass or more, more preferably 70% by mass or more, and still more preferably 75% by mass or more. The upper limit value of the content of the monomer represented by General Formula (1) is not particularly limited, and can be set to, for example, 99% by mass or less, and is preferably 95% by mass or less, more preferably 90% by mass or less, and still more preferably 85% by mass or less. That is, the content of the monomer represented by General Formula (1) is preferably 65% to 99% by mass, more preferably 70% to 95% by mass, still more preferably 75% to 90% by mass, and particularly preferably 75% to 85% by mass.

As described above, the monomer represented by General Formula (1), in a case of being made into a curable composition, makes it possible to obtain a cured product in which resin chipping in a case of being released from a mold is highly suppressed, and makes it possible to achieve a low viscosity of the curable composition and a high refractive index of the obtained cured product. Moreover, even in a case where the concentration of the monomer represented by General Formula (1) in the curable composition is set to a high concentration, the composition can exhibit excellent temporal stability.

In the present invention, the curable composition can also be made into a curable composition containing the monomer represented by General Formula (1) without containing the difunctional (meth)acrylic acid thioester monomer, and in this case, the content of the monomer represented by General Formula (1) can be set to, for example, 99.99% by mass or less, and is preferably 99.95% by mass or less, more preferably 99.90% by mass or less, and still more preferably 99.7% by mass or less. In this case, the lower limit value of the content of the monomer represented by General Formula (1) can be set to, for example, 97% by mass or more, and is preferably 98% by mass or more and more preferably 99.0% by mass or more. That is, in this case, the content of the monomer represented by General Formula (1) is preferably 97% to 99.99% by mass, more preferably 98% to 99.95% by mass, still more preferably 99.0% to 99.90% by mass, and particularly preferably 99.0% to 99.7% by mass.

The curable composition may contain two or more types of monomers represented by General Formula (1). In a case where the curable composition contains two or more types of monomers represented by General Formula (1), the total content of the two or more types of monomers is preferably within the above-mentioned range.

### <Difunctional (Meth)Acrylic Acid Thioester Monomer>

The curable composition according to the embodiment of the present invention contains a difunctional (meth)acrylic acid thioester monomer as a monomer for dilution, in addition to the above-mentioned monomer represented by General Formula (1).

The difunctional (meth)acrylic acid thioester monomer means a compound having two (meth)acryloylthio groups.

The group that links two (meth)acryloylthio groups (hereinafter, referred to as “linking group LL”) is not particularly limited, but it is preferably a group having no branched structure, and is more preferably, for example, a linear alkylene group, or a group in which, among one —CH₂— or two or more —CH₂—'s not adjacent to each other in a linear alkylene group, the —CH₂— not adjacent to an S atom is substituted with —S—.

The number of carbon atoms in the linear alkylene group that can be taken as the linking group LL is preferably 1 to 9, more preferably 3 to 7, and still more preferably 3 to 5.

In the present invention, the “group in which, among one —CH₂— or two or more —CH₂—'s not adjacent to each other in a linear alkylene group, the —CH₂— not adjacent to an S atom is substituted with —S—” means, in other words, a group which has an —S— bond in a linear alkylene group, which does not have a structure in which two or more S atoms are linked (for example, a disulfide structure or a polysulfide structure in which three or more S atoms are linked), and in which a bonding site to a (meth)acryloylthio group is —CH₂—.

In the group in which, among one —CH₂— or two or more —CH₂—'s not adjacent to each other in a linear alkylene group, the —CH₂— not adjacent to an S atom is substituted with —S—, the description of the linear alkylene group that can be taken as the linking group LL can be preferably applied to the linear alkylene group before —CH₂— is substituted with —S—.

The molecular weight of the difunctional (meth)acrylic acid thioester monomer is preferably 150 to 400 and more preferably 200 to 350.

Hereinafter, preferred specific examples of the difunctional (meth)acrylic acid thioester monomer are listed, but the present invention is not limited to these compounds.

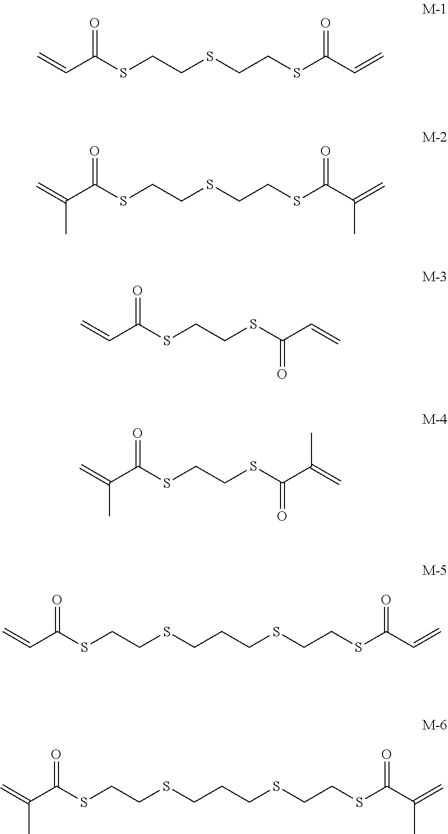

Among these compounds, the above-mentioned exemplary compound M-1 or M-2 is preferable as the difunctional (meth)acrylic acid thioester monomer.

The method for obtaining the above-mentioned difunctional (meth)acrylic acid thioester monomer is not particularly limited, and the difunctional (meth)acrylic acid thioester monomer may be commercially available or may be synthesized by a common method.

The content of the difunctional (meth)acrylic acid thioester monomer in the curable composition according to the embodiment of the present invention can be set to, for example, 40% by mass or less, and is preferably 35% by mass or less, more preferably 30% by mass or less, and still more preferably 25% by mass or less. The lower limit value of the content of the difunctional (meth)acrylic acid thioester monomer is not particularly limited and can be set to, for example, 1% by mass or more, and it is preferably 5% by mass or more, more preferably 10% by mass or more, and still more preferably 15% by mass or more. That is, the content of the difunctional (meth)acrylic acid thioester monomer is preferably 1% to 40% by mass, more preferably 5% to 35% by mass, still more preferably 10% to 30% by mass, and particularly preferably 15% to 25% by mass.

By controlling the amount of the difunctional (meth)acrylic acid thioester monomer in the curable composition, it is possible to adjust the function of relieving stress in a case where the cured product undergoes thermal changes.

The curable composition according to the embodiment of the present invention may contain two or more types of difunctional (meth)acrylic acid thioester monomers. In a case where two or more types of difunctional (meth)acrylic acid thioester monomers are contained, the total content of the two or more types of difunctional (meth)acrylic acid thioester monomers is preferably within the above-mentioned range.

### <Other Components>

The curable composition according to the embodiment of the present invention may further contain other components in addition to the monofunctional (meth)acrylic acid thioester monomer represented by General Formula (1) and the difunctional (meth)acrylic acid thioester monomer. Examples of the other components include a photoradical polymerization initiator.

### (Photoradical Polymerization Initiator)

The curable composition according to the embodiment of the present invention preferably contains a photoradical polymerization initiator. The curable composition according to the embodiment of the present invention makes it possible to obtain a cured product exhibiting a high refractive index by photopolymerization due to the action of the photoradical polymerization initiator. As the photoradical polymerization initiator, a compound that is commonly used as a photoradical polymerization initiator can be used appropriately according to the conditions of a photopolymerization (photocuring) step which will be described later, and specifically, the following compounds can be used.

Examples of the photoradical polymerization initiator compounds include bis(2,6-dimethoxybenzoyl)-2,4,4-trimethylpentyl phosphine oxide, bis(2,6-dimethylbenzoyl)-2,4,4-trimethylpentyl phosphine oxide, bis(2,4,6-trimethylbenzoyl)-2,4,4-trimethylpentyl phosphine oxide, bis(2,6-dichlorobenzoyl)-2,4,4-trimethylpentyl phosphine oxide, 1-phenyl-2-hydroxy-2-methylpropan-1-one, 1-hydroxycyclohexylphenylketone, 1-(4-isopropylphenyl)-2-hydroxy-2-methylpropan-1-one, 1,2-diphenylethanedione, methylphenyl glyoxylate, 1-[4-(2-hydroxyethoxy)-phenyl]-2-hydroxy-2-methyl-1-propan-1-one, 2-hydroxy-1-{4-[4-(2-hydroxy-2-methyl-propionyl)-benzyl]phenyl}-2-methyl-propan-1-one, 2,2-dimethoxy-1,2-diphenylethan-1-one, 2-methyl-1-(4-methylthiophenyl)-2-morpholinopropan-1-one, 2-benzyl-2-dimethylamino-1-(4-morpholinophenyl)-butanone-1,2,4,6-trimethylbenzoyl-diphenyl-phosphine oxide, and bis(2,4,6-trimethylbenzoyl)-phenylphosphine oxide.

Above all, in the present invention, 1-hydroxycyclohexylphenylketone (available as Irgacure 184 (trade name) from BASF SE), bis(2,4,6-trimethylbenzoyl)-phenylphosphine oxide (available as Irgacure 819 (trade name) from BASF SE), 2,4,6-trimethylbenzoyl-diphenyl-phosphine oxide (available as Irgacure TPO (trade name) from BASF SE), 2,2-dimethoxy-1,2-diphenylethan-1-one (available as Irgacure 651 (trade name) from BASF SE), 1-[4-(2-hydroxyethoxy)-phenyl]-2-hydroxy-2-methyl-1-propan-1-one, or 2-methyl-1-(4-methylthiophenyl)-2-morpholinopropan-1-one can be preferably used as the photoradical polymerization initiator.

In a case where the curable composition contains a photoradical polymerization initiator, the content of the photoradical polymerization initiator in the curable composition is preferably 0.01% to 5.0% by mass, more preferably 0.05% to 1.0% by mass, and still more preferably 0.05% to 0.5% by mass.

As long as it does not go against the spirit of the present invention, the curable composition containing the monomer represented by General Formula (1) may contain a polymer or a monomer other than the above-mentioned components, a dispersant, a plasticizer, a heat stabilizer, a mold release agent, a solvent, and the like.

In a case where the curable composition is used as a material for imprinting, it is preferable that the content of inorganic particles in the curable composition is kept to 30% by mass or less, and it is more preferable that the curable composition does not contain the inorganic particles.

From the viewpoint of improving handleability in a case of molding a cured product, in particular improving the followability to a mold in a case of carrying out imprinting, and forming a cured product of higher quality, the viscosity of the curable composition containing the monomer represented by General Formula (1) is preferably less than 170 mPa·s, more preferably less than 135 mPa·s, still more preferably less than 120 mPa·s, even still more preferably less than 100 mPa·s, particularly preferably less than 65 mPa·s, and most preferably less than 55 mPa·s. The lower limit value of the viscosity of the curable composition is not particularly limited and is practically 1 mPa·s or more.

The viscosity of the curable composition is a viscosity measured by the method described in Examples which will be given later. It is noted that 1 mPa·s is 1 cP.

The refractive index of the liquid of the curable composition according to the embodiment of the present invention can be evaluated using a refractive index nD at 25° C. and a wavelength of 589 nm (in the present invention, also simply referred to as “refractive index nD of the liquid of the curable composition” or “refractive index nD of the liquid at 25° C.”).

The refractive index nD of the liquid of the curable composition according to the embodiment of the present invention can be 1.650 or more and is preferably 1.660 or more. The upper limit value of the refractive index nD of the liquid of the curable composition according to the embodiment of the present invention is not particularly limited and is practically 1.750 or less.

The refractive index nD of the liquid of the curable composition is a value measured using an Abbe refractometer (for example, trade name: multi-wavelength Abbe refractometer DR-M2 or DR-M4, manufactured by Atago Co., Ltd.), and specifically, a sample for measurement (curable composition) can be prepared and measured according to the description of Examples which will be given later. In addition, Japan Industrial Standards (JIS) B 7090:1999 Optics and optical instruments-Reference wavelengths (International Organization for Standardization (ISO) 7944: 1998 Optics and optical instruments-Reference wavelengths) can be referred to as appropriate.

The curable composition according to the embodiment of the present invention can be used for manufacturing a cured product that is required to have a high refractive index.

Above all, the curable composition according to the embodiment of the present invention has a low viscosity, excellent temporal stability, and suppressed resin chipping in a case of being released from a mold, and thus can be preferably used as a material for imprinting such as nanoimprinting, and can be preferably used for manufacturing a cured product exhibiting a high refractive index.

### Cured Product

The cured product according to the embodiment of the present invention is a cured product obtained by curing the curable composition containing the monomer represented by General Formula (1) and the difunctional (meth)acrylic acid thioester monomer.

The cured product according to the embodiment of the present invention is obtained by carrying out a polymerization reaction of a monomer containing the monomer represented by General Formula (1) and the difunctional (meth)acrylic acid thioester monomer, followed by curing. The cured product according to the embodiment of the present invention may contain unreacted monomers (for example, the monomer represented by General Formula (1) and the difunctional (meth)acrylic acid thioester monomer) and the like.

As described above, the cured product according to the embodiment of the present invention can exhibit a high refractive index.

The refractive index of the cured product can be evaluated using a refractive index nD at 25° C. and a wavelength of 589 nm (in the present invention, also simply referred to as “refractive index nD of the cured product” or “refractive index nD of the cured product obtained by curing the curable composition”).

The refractive index nD of the cured product according to the embodiment of the present invention can be 1.670 or more, and is preferably 1.680 or more, more preferably 1.690 or more, still more preferably 1.700 or more, and particularly preferably 1.710 or more. In a region where the refractive index nD of the cured product is 1.670 or more, a difference in the order of 10⁻³ of the refractive index nD results in a difference in the performance of high refractive index in a case of being applied to an optical member. For example, a difference of 0.004 between a refractive index nD of 1.686 of a cured product of No. 101 and a refractive index nD of 1.690 of each of cured products of No. 102 and No. 103 in Examples which will be given later can be said to be a significant difference.

The upper limit value of the refractive index nD of the cured product according to the embodiment of the present invention is not particularly limited and is practically 1.800 or less.

The refractive index nD of the cured product is a value measured using an Abbe refractometer (for example, trade name: multi-wavelength Abbe refractometer DR-M2 or DR-M4, manufactured by Atago Co., Ltd.), and specifically, a sample for measurement (cured product) can be produced and measured according to the description of Examples which will be given later. In a case of forming the cured product, a heating step may be employed instead of an ultraviolet irradiating step described in Examples which will be given later, or both of the heating step and the ultraviolet irradiating step may be employed. In addition, JIS B 7090: 1999 Optics and optical instruments-Reference wavelengths (ISO 7944: 1998 Optics and optical instruments-Reference wavelengths) can be referred to as appropriate.

The transmittance of the cured product according to the embodiment of the present invention over the entire visible light wavelength range of 360 to 830 nm is preferably 85% or more, more preferably 90% or more, and still more preferably 95% or more. In addition, the upper limit value of the transmittance of the cured product according to the embodiment of the present invention is not particularly limited and is practically 99% or less.

The transmittance of the cured product is a value of an external transmittance including surface reflection, which is measured using an ultraviolet-visible spectrophotometer (for example, UV-2600 (trade name), manufactured by Shimadzu Corporation) for a cured product having a thickness of 1 mm.

### Manufacturing Method of Cured Product

The cured product according to the embodiment of the present invention can be manufactured by a method including a step of photocuring the above-mentioned curable composition. In a case of photocuring, it is preferable that the above-mentioned photoradical polymerization initiator is contained in the curable composition.

With regard to the conditions for photocuring, the description of light irradiation in a diffractive optical element which will be described later can be preferably applied.

In the manufacturing method of the cured product according to the embodiment of the present invention, it is preferable that the curable composition according to the embodiment of the present invention is photocured while being pressed against a mold to obtain a cured product to which a pattern of the mold has been transferred.

From the viewpoint of peelability, a mold that has been subjected to a surface treatment with chromium nitride is preferable as the mold. With regard to the surface treatment of a mold with chromium nitride, for example, the description of the treatment with chromium nitride in paragraph [0108] of WO2019/044863A can be applied as it is, except that “metal mold” is replaced with “mold”.

In addition, the cured product according to the embodiment of the present invention can be manufactured by an imprinting technology using the curable composition according to the embodiment of the present invention while suppressing resin chipping to a sufficient level in a case of being released from a mold.

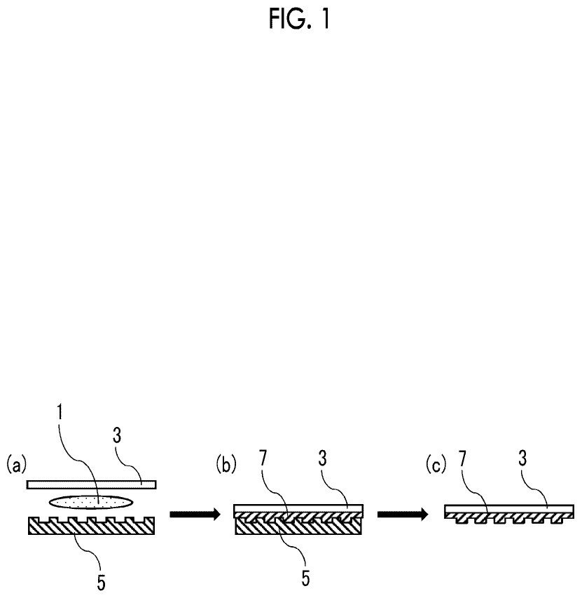

FIG. 1 is a schematic cross-sectional view schematically showing a method of manufacturing the cured product according to the embodiment of the present invention by an imprinting technology using the curable composition according to the embodiment of the present invention. The size, shape, thickness, and the like of a substrate 3, the size, shape, transfer pattern, thickness, and the like of a mold 5, the amount of a curable composition 1 used, and the like can be appropriately adjusted so that a cured product 7 having a desired size, shape, transferred pattern, thickness, and the like is obtained.

As shown in FIG. 1, the cured product according to the embodiment of the present invention can be manufactured in such a manner that (a) first, the curable composition 1 is sandwiched between the mold 5 and the substrate 3, (b) the curable composition 1 is pressed against the mold 5 in a state of being embossed and the curable composition 1 is photocured by ultraviolet irradiation (UV irradiation) to produce the cured product 7 to which a pattern of the mold 5 is transferred, and (c) the obtained cured product 7 and the substrate 3 are released (peeled off) from the mold 5.

The ultraviolet irradiation is not particularly limited as long as the curable composition 1 is cured, and may be carried out from any side of the mold 5 or the substrate 3. It is preferable that the substrate 3 is transparent and the irradiation is carried out from the substrate 3 side.

In addition, the obtained cured product 7 may be used in a form integrated with the substrate 3 or may be used in a form consisting of the cured product 7 after the substrate 3 is peeled off.

With regard to the substrate 3, the description of the transparent substrate in the diffractive optical element which will be described later can be preferably applied.

### Applications of Cured Product

The cured product according to the embodiment of the present invention can be used in a variety of applications, and since the cured product exhibits a high refractive index, it can be preferably used for an optical material, and above all, it can be preferably used for a diffractive optical element. The diffractive optical element according to the embodiment of the present invention can also be used, for example, as a diffractive optical element for a waveguide in augmented reality glasses (AR glasses).

### Diffractive Optical Element

The diffractive optical element according to the embodiment of the present invention is a diffractive optical element including a surface which has a diffraction grating shape and is formed of the cured product according to the embodiment of the present invention, and is formed by curing the curable composition according to the embodiment of the present invention.

The diffractive optical element according to the embodiment of the present invention preferably has a maximum thickness of 0.05 μm to 100 μm. The maximum thickness of the diffractive optical element is more preferably 0.1 μm to 50 μm and still more preferably 0.2 μm to 30 μm. In addition, a level difference (lattice thickness) of the diffraction grating shape (periodic structure) of the diffractive optical element is preferably 0.05 μm to 100 μm, more preferably 0.05 μm to 50 μm, and still more preferably 0.1 μm to 30 μm. Further, a pitch of the diffraction grating shape of the diffractive optical element may be 0.05 μm to 1 mm, and is preferably 0.05 μm to 100 μm and preferably varies within the same diffractive optical element depending on the required optical aberration.

The diffractive optical element can be manufactured, for example, by the following procedure.

The curable composition is sandwiched between a transparent substrate and a surface of a mold such as a metal mold, which has a surface processed into a diffraction grating shape. Thereafter, the curable composition may be pressurized and stretched to a desired extent. In the sandwiched state, the curable composition is cured by irradiation with light from the transparent substrate side. The cured product is then released from the mold such as a metal mold. After the release from the mold, the cured product may be further irradiated with ultraviolet rays from a side opposite to the transparent substrate side.

Examples of the transparent substrate include flat glass such as BK glass, and a flat transparent resin (a (meth)acrylic resin, a polycarbonate resin, polyethylene terephthalate, or the like). The surface of the transparent substrate may be subjected to a surface treatment such as an ozone treatment.

The transparent substrate used in the above-mentioned manufacturing may be included in the diffractive optical element as it is, or may be peeled off.

The light used for the light irradiation to cure the curable composition is preferably ultraviolet rays or visible rays and more preferably ultraviolet rays. For example, a metal halide lamp, a low pressure mercury lamp, a high pressure mercury lamp, an ultra-high pressure mercury lamp, a germicidal lamp, a xenon lamp, and a light emitting diode (LED) light source lamp are suitably used. The illuminance of ultraviolet light used for the light irradiation to cure the curable composition is preferably 1 to 100 mW/cm², more preferably 1 to 75 mW/cm², and still more preferably 5 to 50 mW/cm². The curable composition may be irradiated multiple times with ultraviolet light having different illuminance. The exposure amount of the ultraviolet light is preferably 0.4 to 10 J/cm², more preferably 0.5 to 5 J/cm², and still more preferably 1 to 3 J/cm². The atmosphere during the light irradiation is preferably air or an inert gas-purged atmosphere, and more preferably an atmosphere in which air has been purged with nitrogen until the oxygen concentration reaches 1% or less.

Using the curable composition according to the embodiment of the present invention as a material for imprinting, the diffraction grating shape can be produced in such a manner that the material is pressed against a mold having a desired pattern with a size of nanometers to several hundred micrometers in a state of being embossed, followed by photocuring to produce the cured product according to the embodiment of the present invention to which the pattern of the mold has been transferred, and then the cured product is released from the mold.

With regard to the conditions for the photocuring, the description in the above-mentioned manufacturing method of a cured product and the description in the above-mentioned manufacturing method of a diffractive optical element can be applied.

For the points other than the above-mentioned points, the description of the imprinting in common use can be adopted without any particular limitation, and for example, a nanoimprint technology handbook (edited by the Japan Society of Applied Physics and Nanoimprint Technology Research Association, published by Ohmsha, Ltd., Dec. 1, 2019) can be referred to.

### EXAMPLES

Hereinafter, the present invention will be described in more detail with reference to Examples. The materials, amounts used, proportions, treatment details, treatment procedures, and the like shown in the Examples below can be modified as appropriate without departing from the spirit of the present invention. Therefore, the scope of the present invention should not be construed as being limited to the specific examples shown below. In the following description, room temperature means 25° C. unless otherwise specified.

All steps from the preparation of the curable composition to the production or evaluation test of the cured product were carried out in an environment where a yellow lamp was used as lighting.

### Synthesis Example

The monomer represented by General Formula (1) was synthesized as follows.

### Synthesis Example 1: Synthesis of Compound (A-1)

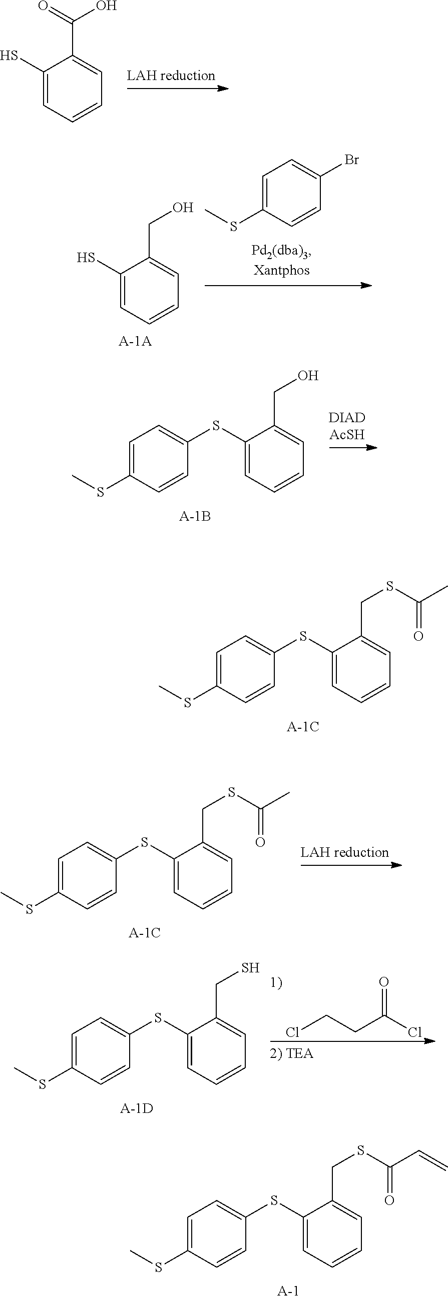

<Synthesis of Compound (A-1A)>9.23 g (243.2 mmol) of lithium aluminum hydride (LAH) and 450 mL of tetrahydrofuran (hereinafter, abbreviated as “THF”) were mixed while being purged with nitrogen, and then the mixture was cooled such that the internal temperature (liquid temperature) was 0° C. A mixed solution of 25.0 g (162.1 mmol) of thiosalicylic acid and 283mL of THF was added dropwise thereto such that the liquid temperature did not exceed 7° C., and then the mixture was heated such that the internal temperature (liquid temperature) was 25° C. After stirring for 1 hour, the mixture was cooled to 0° C., and 500 mL of ethyl acetate, 50 mL of concentrated hydrochloric acid, and 500 mL of water were each added dropwise thereto in this order, followed by stirring and then washing and liquid separation. Next, 500 mL of water was added thereto, followed by stirring and then washing and liquid separation. Dehydration with magnesium sulfate, filtration, and concentration were carried out to obtain 22.3 g of an oily compound (A-1A). The yield was 98%.

### <Synthesis of Compound (A-1B)>

20.0 g (142.7 mmol) of the compound (A-1A), 27.6 g (135.9 mmol) of 4-bromothioanisole, 204 mL of N,N-dimethylacetamide (DMAc), and 36.1 g (278.6 mmol) of N,N-diisopropylethylamine (DIPEA) were mixed while being purged with nitrogen, and then 3.11 g (3.40 mmol) of tris(dibenzylideneacetone) dipalladium (Pd₂(dba)₃) and 3.93 g (6.80 mmol) of 4,5-bis(diphenylphosphino)-9,9-dimethylxanthene (Xantphos) were added thereto, and the mixture was heated such that the internal temperature (liquid temperature) was 120° C. After stirring for 5 hours, 800 mL of ethyl acetate and 800 mL of 1 N hydrochloric acid were added thereto, and the insoluble matter was filtered, followed by washing and liquid separation. Next, 800 mL of a 5% sodium hydrogen carbonate aqueous solution was added thereto, followed by stirring and then washing and liquid separation. Dehydration with magnesium sulfate, filtration, and concentration were carried out to obtain an oily composition which was then purified by column chromatography to obtain 27.6 g of a compound (A-1B). The yield was 74%.

### <Synthesis of Compound (A-1C)>

20.3 g (77.6 mmol) of triphenylphosphine (PPh₃) and 370 mL of THF were mixed while being purged with nitrogen, and then the mixture was cooled such that the internal temperature (liquid temperature) was 0° C. 15.7 g (77.6 mmol) of diisopropyl azodicarboxylate (DIAD) was added dropwise thereto, followed by stirring for 30 minutes while maintaining the temperature at 0° C. Then, a mixed solution of 18.5 g (70.5 mmol) of the compound (A-1B), 5.90 g (77.6 mmol) of thioacetic acid (AcSH), and 370 mL of THF was added dropwise thereto such that the liquid temperature did not exceed 7° C., followed by stirring for 1 hour. After heating to 25° C., 760 mL of ethyl acetate and 500 mL of a 5% sodium hydrogen carbonate aqueous solution were added thereto, followed by stirring and then washing and liquid separation. Dehydration with magnesium sulfate, filtration, and concentration were carried out to obtain an oily composition which was then purified by column chromatography to obtain 19.7 g of a compound (A-1C). The yield was 87%.

### <Synthesis of Compound (A-1D)>

3.51 g (92.4 mmol) of lithium aluminum hydride (LAH) and 333 mL of THF were mixed while being purged with nitrogen, and then the mixture was cooled such that the internal temperature (liquid temperature) was 0° C. A mixed solution of 18.5 g (57.7 mmol) of the compound (A-1C) and 210 mL of THF was added dropwise thereto such that the liquid temperature did not exceed 7° C., and then the mixture was heated such that the internal temperature (liquid temperature) was 25° C. After stirring for 1 hour, the mixture was cooled to 0° C., and 370 mL of ethyl acetate, 18.5 mL of concentrated hydrochloric acid, and 370 mL of water were each added dropwise thereto in this order, followed by stirring and then washing and liquid separation. Next, 370 mL of water was added thereto, followed by stirring and then washing and liquid separation. Dehydration with magnesium sulfate, filtration, and concentration were carried out to obtain an oily composition which was then purified by column chromatography to obtain 15.6 g of a compound (A-1D). The yield was 97%.

### <Synthesis of Compound (A-1)>

8.00 g (28.7 mmol) of the compound (A-1D) and 204 mL of N,N-dimethylacetamide (DMAc) were mixed while being cooled such that the internal temperature (liquid temperature) was 0° C. 4.01 g (31.6 mmol) of 3-chloropropionyl chloride (3CPC) was added dropwise thereto such that the liquid temperature did not exceed 7° C., and the mixture was heated such that the internal temperature (liquid temperature) was 25° C. After stirring for 1 hour, the mixture was cooled to 0° C., 6.98 g (68.9 mmol) of triethylamine (TEA) was added dropwise thereto such that the liquid temperature did not exceed 7° C., and the mixture was heated such that the internal temperature (liquid temperature) was 25° C. After stirring for 1 hour, 240 mL of ethyl acetate and 800 mL of 1 N hydrochloric acid were added thereto, and the insoluble matter was filtered, followed by washing and liquid separation. Next, 500 mL of a 5% sodium hydrogen carbonate aqueous solution was added thereto, followed by stirring and then washing and liquid separation. Dehydration with magnesium sulfate, filtration, and concentration were carried out to obtain an oily composition which was then purified by column chromatography to obtain 6.57 g of a compound (A-1). The yield was 69%.

¹H-NMR (300 MHz, CDCl₃): δ (ppm) 2.48 (s, 3H), 4.37 (s, 2H), 5.60-5.70 (m, 1H), 6.2-6.5 (m, 2H), 7.1-7.3 (m, 7H), 7.4-7.5 (m, 1H)

### Synthesis Example 2: Synthesis of Compound (A-18)

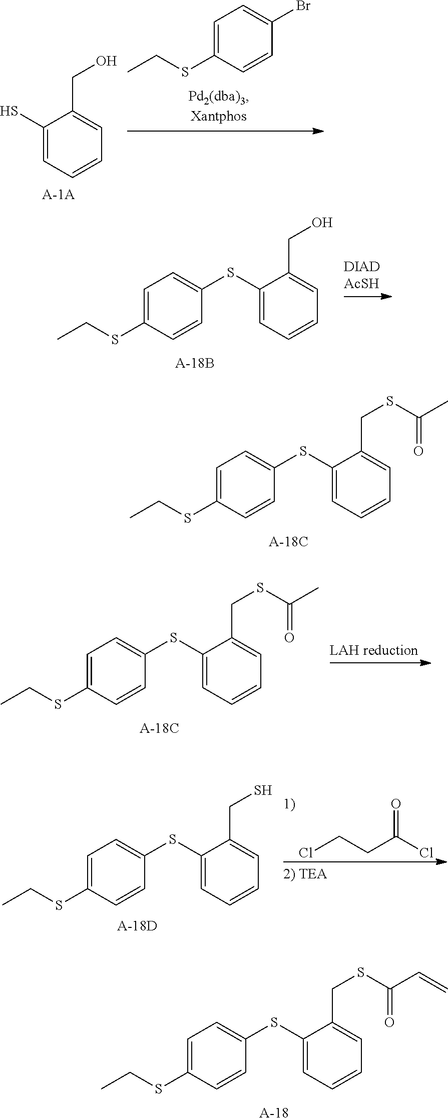

### <Synthesis of Compound (A-18B)>

A compound (A-18B) was synthesized (yield: 70%) in the same manner as in the synthesis of the compound (A-1B), except that 4-bromothioanisole was changed to 1-bromo-4-(ethylthio)benzene.

### <Synthesis of Compound (A-18C)>

A compound (A-18C) was synthesized (yield: 85%) in the same manner as in the synthesis of the compound (A-1C), except that the compound (A-1B) was changed to the compound (A-18B).

### <Synthesis of Compound (A-18D)>

A compound (A-18D) was synthesized (yield: 97%) in the same manner as in the synthesis of the compound (A-1D), except that the compound (A-1C) was changed to the compound (A-18C).

### <Synthesis of Compound (A-18)>

A compound (A-18) was synthesized (yield: 65%) in the same manner as in the synthesis of the compound (A-1), except that the compound (A-1D) was changed to the compound (A-18D).

¹H-NMR (300 MHz, CDCl₃): δ (ppm) 1.25 (t, 3H), 2.66 (d, 2H), 4.37 (s, 2H), 5.6-5.7 (m, 1H), 6.2-6.5 (m, 2H), 7.1-7.3 (m, 7H), 7.4-7.5 (m, 1H)

### Synthesis Example 3: Synthesis of Compound (A-3)

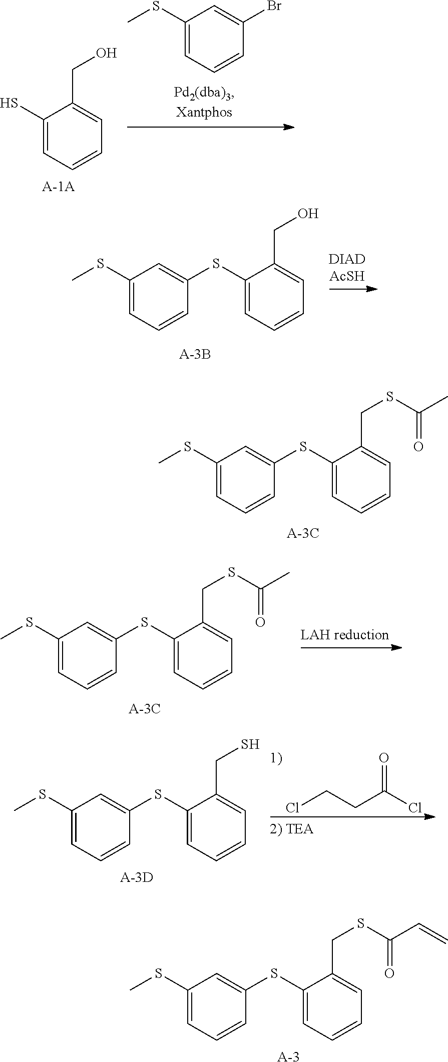

### <Synthesis of Compound (A-3B)>

A compound (A-3B) was synthesized (yield: 74%) in the same manner as in the synthesis of the compound (A-1B), except that 4-bromothioanisole was changed to 3-bromothioanisole.

### <Synthesis of Compound (A-3C)>

A compound (A-3C) was synthesized (yield: 87%) in the same manner as in the synthesis of the compound (A-1C), except that the compound (A-1B) was changed to the compound (A-3B).

### <Synthesis of Compound (A-3D)>

A compound (A-3D) was synthesized (yield: 95%) in the same manner as in the synthesis of the compound (A-1D), except that the compound (A-1C) was changed to the compound (A-3C).

### <Synthesis of Compound (A-3)>

A compound (A-3) was synthesized (yield: 62%) in the same manner as in the synthesis of the compound (A-1), except that the compound (A-1D) was changed to the compound (A-3D).

¹H-NMR (300 MHz, CDCl₃): δ (ppm) 2.41 (s, 3H), 4.37 (s, 2H), 5.6-5.7 (m, 1H), 6.2-6.5 (m, 2H), 6.90 (s, 1H), 7.0-7.2 (m, 3H), 7.3-7.6 (m, 4H)

### Synthesis Example 4: Synthesis of Compound (A-8)

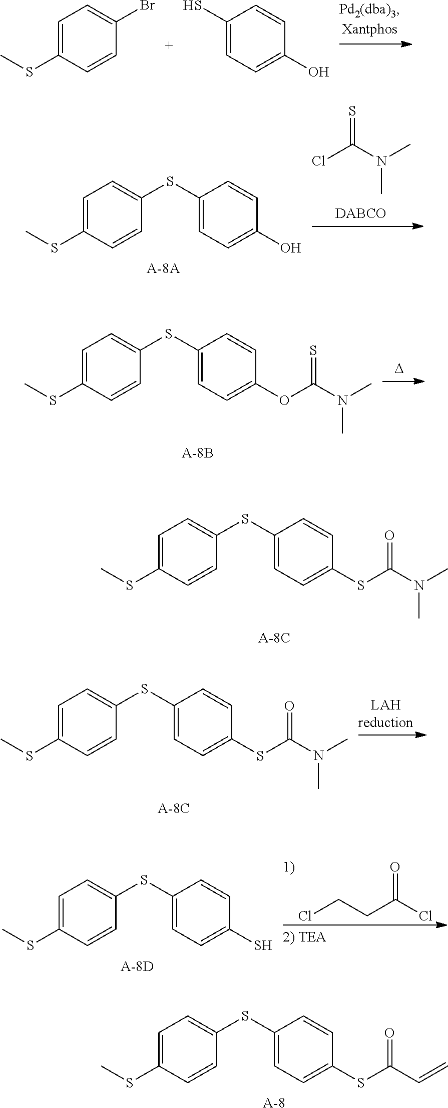

### <Synthesis of Compound (A-8A)>

16.3 g (129.2 mmol) of 4-hydroxybenzenethiol, 25.0 g (123.1 mmol) of 4-bromothioanisole, 185 mL of N,N-dimethylacetamide (DMAc), and 32.6 g (252.3 mmol) of N,N-diisopropylethylamine (DIPEA) were mixed while being purged with nitrogen, and then 2.82 g (3.08 mmol) of tris(dibenzylideneacetone) dipalladium (Pd₂(dba)₃) and 3.56 g (6.16 mmol) of 4,5-bis(diphenylphosphino)-9,9-dimethylxanthene (Xantphos) were added thereto, followed by heating such that the internal temperature (liquid temperature) was 120° C. After stirring for 5 hours, 800 mL of ethyl acetate and 800 mL of 1 N hydrochloric acid were added thereto, and the insoluble matter was filtered, followed by washing and liquid separation. Next, 800 mL of a 5% sodium hydrogen carbonate aqueous solution was added thereto, followed by stirring and then washing and liquid separation. Dehydration with magnesium sulfate, filtration, and concentration were carried out to obtain an oily composition which was then purified by column chromatography to obtain 23.5 g of a compound (A-8A). The yield was 77%.

### <Synthesis of Compound (A-8B)>

22.0 g (88.6 mmol) of the compound (A-8A), 185 mL of N,N-dimethylacetamide (DMAc), 14.9 g (132.9 mmol) of 1,4-diazabicyclo[2.2.2]octane (DABCO), and 12.1 g of N,N-dimethylthiocarbamoyl chloride were mixed while being heated such that the internal temperature (liquid temperature) was 50° C. 240 mL of ethyl acetate and 500 mL of 1 N hydrochloric acid were added thereto, followed by washing and liquid separation. Next, 500 mL of a 5% sodium hydrogen carbonate aqueous solution was added thereto, followed by stirring and then washing and liquid separation. Dehydration with magnesium sulfate, filtration, and concentration were carried out to obtain an oily composition which was then purified by column chromatography to obtain 23.8 g of a compound (A-8B). The yield was 80%.

### <Synthesis of Compound (A-8C)>

23.0 g (68.5 mmol) of the compound (A-8B) was heated at 240° C. for 24 hours and then cooled to room temperature. Ethyl acetate was added thereto, and the dissolved portion was taken out and then concentrated to obtain 19.6 g of a compound (A-8C). The yield was 85%.

### <Synthesis of Compound (A-8D)>

3.39 g (89.4 mmol) of lithium aluminum hydride (LAH) and 360 mL of THF were mixed while being purged with nitrogen, and then the mixture was cooled such that the internal temperature (liquid temperature) was 0° C. A mixed solution of 20.0 g (59.6 mmol) of the compound (A-8C) and 227 mL of THF was added dropwise thereto such that the liquid temperature did not exceed 7° C., and then the mixture was heated such that the internal temperature (liquid temperature) was 25° C. After stirring for 1 hour, the mixture was cooled to 0° C., and 300 mL of ethyl acetate, 17.9 mL of concentrated hydrochloric acid, and 370 mL of water were each added dropwise thereto in this order, followed by stirring and then washing and liquid separation. Next, 370 mL of water was added thereto, followed by stirring and then washing and liquid separation. Dehydration with magnesium sulfate, filtration, and concentration were carried out to obtain an oily composition which was then purified by column chromatography to obtain 15.1 g of a compound (A-8D). The yield was 96%.

### <Synthesis of Compound (A-8)>

15.0 g (56.7 mmol) of the compound (A-8D) and 29.6 mL of N,N-dimethylacetamide (DMAc) were mixed while being cooled such that the internal temperature (liquid temperature) was 0° C. 7.92 g (62.4 mmol) of 3-chloropropionyl chloride (3CPC) was added dropwise thereto such that the liquid temperature did not exceed 7° C., and the mixture was heated such that the internal temperature (liquid temperature) was 25° C. After stirring for 1 hour, the mixture was cooled to 0° C., 13.8 g (136.1 mmol) of triethylamine (TEA) was added dropwise thereto such that the liquid temperature did not exceed 7° C., and the mixture was heated such that the internal temperature (liquid temperature) was 25° C. After stirring for 1 hour, 150 mL of ethyl acetate and 200 mL of 1 N hydrochloric acid were added thereto, followed by washing and liquid separation. Next, 500 mL of a 5% sodium hydrogen carbonate aqueous solution was added thereto, followed by stirring and then washing and liquid separation. Dehydration with magnesium sulfate, filtration, and concentration were carried out to obtain an oily composition which was then purified by column chromatography to obtain 11.7 g of a compound (A-8). The yield was 65%.

¹H-NMR (300 MHz, CDCl₃): δ (ppm) 2.50 (s, 3H), 5.7-5.8 (m, 1H), 6.3-6.5 (m, 2H), 7.1-7.3 (m, 6H), 7.3-7.4 (m, 2H)

### Synthesis Example 5: Synthesis of Compound (A-6)

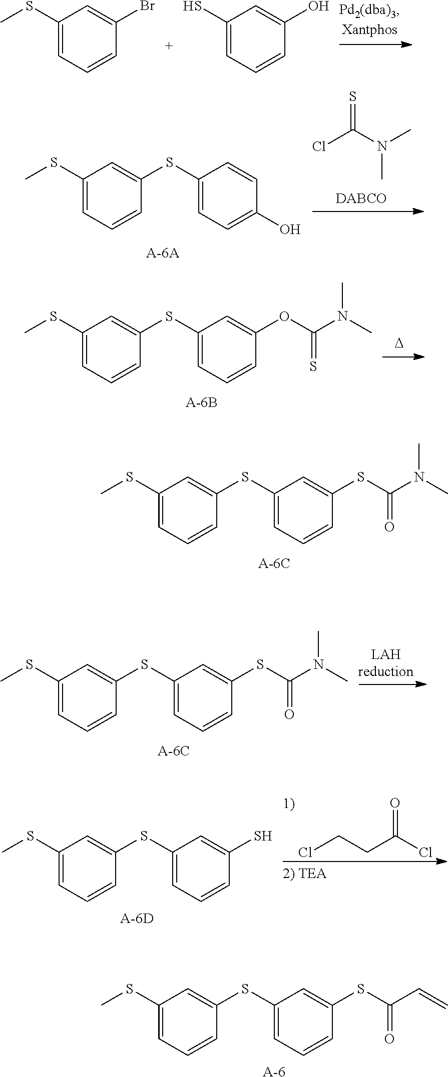

### <Synthesis of Compound (A-6A)>

A compound (A-6A) was synthesized (yield 72%) in the same manner as in the synthesis of the compound (A-8A), except that 4-bromothioanisole was changed to 3-bromothioanisole and 4-hydroxybenzenethiol was changed to 3-hydroxybenzenethiol.

### <Synthesis of Compound (A-6B)>

A compound (A-6B) was synthesized (yield: 82%) in the same manner as in the synthesis of the compound (A-8B), except that the compound (A-8A) was changed to the compound (A-6A).

### <Synthesis of Compound (A-6C)>

A compound (A-6C) was synthesized (yield: 85%) in the same manner as in the synthesis of the compound (A-8C), except that the compound (A-8B) was changed to the compound (A-6B).

### <Synthesis of Compound (A-6D)>

A compound (A-6D) was synthesized (yield: 85%) in the same manner as in the synthesis of the compound (A-8D), except that the compound (A-8C) was changed to the compound (A-6C).

### <Synthesis of Compound (A-6)>

A compound (A-6) was synthesized (yield: 66%) in the same manner as in the synthesis of the compound (A-8), except that the compound (A-8D) was changed to the compound (A-6D).

¹H-NMR (300 MHz, CDCl₃): δ (ppm) 2.48 (s, 3H), 5.7-5.8 (m, 1H), 6.3-6.5 (m, 2H), 7.0-7.4 (m, 8H)

### Synthesis Example 6: Synthesis of Compound (A-9)

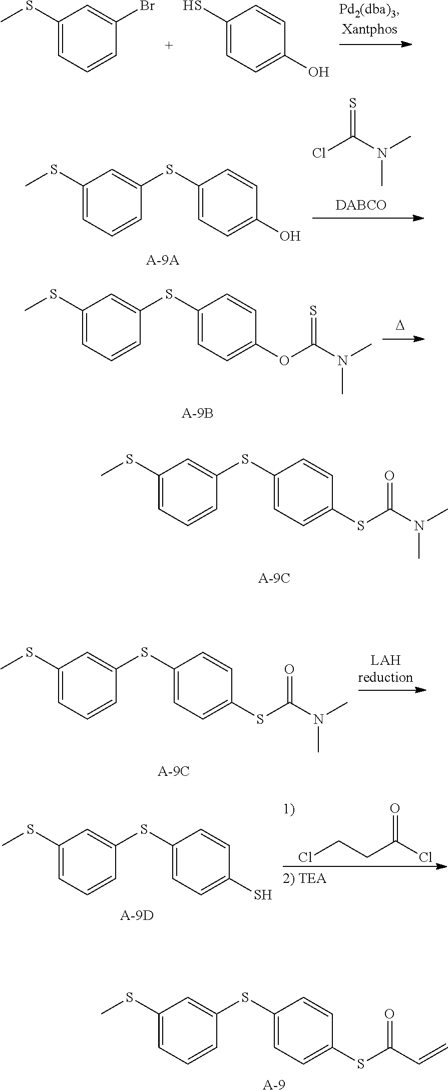

### <Synthesis of Compound (A-9A)>

A compound (A-9A) was synthesized (yield: 70%) in the same manner as in the synthesis of the compound (A-8A), except that 4-bromothioanisole was changed to 3-bromothioanisole.

### <Synthesis of Compound (A-9B)>

A compound (A-9B) was synthesized (yield: 83%) in the same manner as in the synthesis of the compound (A-8B), except that the compound (A-8A) was changed to the compound (A-9A).

### <Synthesis of Compound (A-9C)>

A compound (A-9C) was synthesized (yield: 85%) in the same manner as in the synthesis of the compound (A-8C), except that the compound (A-8B) was changed to the compound (A-9B).

### <Synthesis of Compound (A-9D)>

A compound (A-9D) was synthesized (yield: 83%) in the same manner as in the synthesis of the compound (A-8D), except that the compound (A-8C) was changed to the compound (A-9C).

### <Synthesis of Compound (A-9)>

A compound (A-9) was synthesized (yield: 63%) in the same manner as in the synthesis of the compound (A-8), except that the compound (A-8D) was changed to the compound (A-9D).

¹H-NMR (300 MHz, CDCl₃): δ (ppm) 2.48 (s, 3H), 5.7-5.8 (m, 1H), 6.3-6.5 (m, 2H), 7.0-7.4 (m, 8H)

### Synthesis Example 7: Synthesis of Compound (A-16)

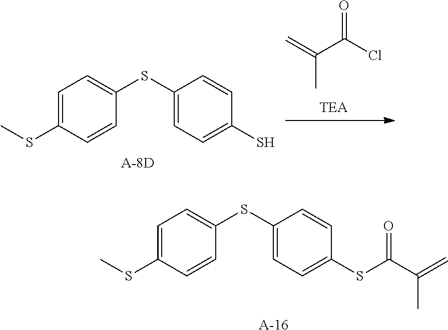

15.0 g (56.7 mmol) of the compound (A-8D), 29.6 mL of N,N-dimethylacetamide (DMAc), and 6.88 g (68.1 mmol) of triethylamine (TEA) were mixed while being cooled such that the internal temperature (liquid temperature) was 0° C. 6.52 g (62.4 mmol) of methacryloyl chloride was added dropwise thereto such that the liquid temperature did not exceed 7° C., and then the mixture was heated such that the internal temperature (liquid temperature) was 25° C. After stirring for 1 hour, 150 mL of ethyl acetate and 200 mL of 1 N hydrochloric acid were added thereto, followed by washing and liquid separation. Next, 500 mL of a 5% sodium hydrogen carbonate aqueous solution was added thereto, followed by stirring and then washing and liquid separation. Dehydration with magnesium sulfate, filtration, and concentration were carried out to obtain an oily composition which was then purified by column chromatography to obtain 8.5 g of a compound (A-16). The yield was 45%.

¹H-NMR (300 MHz, CDCl₃): 8 (ppm) 2.03 (s, 3H), 2.47 (s, 3H), 5.72 (s, 1H), 6.20 (s, 1H), 7.1-7.3 (m, 6H), 7.3-7.4 (m, 2H)

### Example 1: Preparation of Curable Composition

A monomer represented by General Formula (1) or a comparative monomer, a difunctional (meth)acrylic acid thioester monomer, and a photopolymerization initiator were mixed to have the composition shown in Table 1 which will be given later, and the mixture was stirred while being heated to 50° C. to make it homogenous, thereby preparing a curable composition.

Curable composition Nos. 101 to 112 are compositions containing the monomer represented by General Formula (1) according to the embodiment of the present invention, and curable composition Nos. c11 to c14 are comparative compositions not containing the monomer represented by General Formula (1) according to the embodiment of the present invention.

### Example 2: Production of Cured Product

The curable composition prepared above was sandwiched between glass plates subjected to a hydrophobization treatment such that a film thickness of a cured product was 150 μm, irradiated with ultraviolet (UV) rays using a UV irradiation device (EXECURE 3000 (trade name), manufactured by HOYA CANDEO OPTRONICS CORPORATION) under conditions of an integrated light amount of 1.2 J/cm² and an illuminance of 5 mW/cm² in an atmosphere purged with nitrogen (N₂) so that the oxygen concentration was 1% or less, and then peeled off from the glass plates to produce a cured product.

### [Evaluation 1] Refractive Index

The refractive index nD of a sample for measurement at a wavelength of 589 nm was measured under a condition of 25° C. by a multi-wavelength Abbe refractometer DR-M2 or DR-M4 (trade name, manufactured by Atago Co., Ltd.). As the sample for measurement, the curable composition prepared above was used for the refractive index nD of the liquid of the composition, and the cured product prepared above was used for the refractive index nD of the cured product. The refractive index nD of the cured product was evaluated according to the following standards.

The results are summarized in Table 1.

### —Evaluation Standards for Refractive Index nD of Cured Product—

A2: nD≥1.710
A1: 1.700≤nD<1.710
B: 1.690≤nD<1.700
C: 1.680≤nD<1.690
D: 1.670≤nD<1.680
E: nD<1.670

### Evaluation 2: Viscosity Measurement

The viscosity η of the curable composition prepared above at a shear rate of 10 s⁻¹ and 25° C. was measured using a rheometer (trade name: Rheostress 6000, manufactured by Thermo Fisher Scientific Inc.), and evaluated according to the following standards. 1 cP is 1 mPa·s.

The results are summarized in Table 1. It is noted that the unit “cP” is omitted from the viscosity values in the table.

### —Evaluation Standards for Viscosity—

A3: η<55 cP
A2: 55 cP≤η<65 cP
A1: 65 cP≤η<100 cP
B2: 100 cP≤η<120 cP
B1: 120 cP≤η<135 cP
C: 135 cP≤η<170 cP
D: 170 cP≤η

### Evaluation 3: Temporal Stability of Liquid

The curable composition prepared above was stored at 25° C. for 2 months and whether or not any change in crystal precipitation and/or turbidity occurred over time was visually observed, and the temporal stability of the liquid was evaluated according to the following standards. The storage was carried out in an environment in which a yellow lamp was used as lighting.

### —Evaluation Standards for Temporal Stability of Liquid—

A2: There was no change in either crystal precipitation or turbidity during the storage period of 2 months, and the liquid remained transparent.
A1: There was no change in either crystal precipitation or turbidity during the storage period of 1 month, and the liquid remained transparent, but a small amount of crystal precipitation was observed during the storage period of more than 1 month and up to 2 months.
N: There was a change in at least one of crystal precipitation or turbidity during the storage period of 1 month.

### Evaluation 4: Defective Product Rate of Metal Mold Release Product

A metal mold made of SUS and having a disk shape with a diameter of 30 mm was prepared, the metal mold having, on one surface, 27 concentric grooves, each having a depth of 20 μm and a width of 50 μm at intervals of 0.5 mm from the center of the circle as patterns for transfer, and the surface being subjected to chromium nitride plating. 10 μL of the curable composition prepared above was placed at the center of an ozone-treated BK-7 glass substrate (diameter: 35 mm), the curable composition was spread to a diameter of 30 mm, and then, in a state where a metal mold was pressed against the curable composition, the curable composition was UV-cured using a UV irradiation device (EXECURE 3000 (trade name), manufactured by HOYA CANDEO OPTRONICS CORPORATION) under conditions of an integrated light amount of 1.2 J/cm² and an illuminance of 5 mW/cm² in an atmosphere purged with nitrogen (N₂) so that the oxygen concentration was 1% or less, and a composite consisting of the glass and a cured product of the curable composition was then removed from the metal mold to obtain a sample. A total of 20 samples were prepared in the same manner. The surface of the cured product of each sample (the surface that had been in contact with the metal mold) was observed with an optical microscope (manufactured by KEYENCE CORPORATION), and a sample in which chipping occurred in the cured product in a range of a radius of 5 mm from the center was evaluated as a defective product. The proportion of defective products among the 20 samples produced was calculated as a defective product rate, and the results were evaluated according to the following standards.

### —Evaluation Standards for Defective Product Rate of Metal Mold Release Product—

A: The defective product rate was 10% or less (the number of defective products was 0 to 2)
B: The defective product rate was more than 10% and 20% or less (the number of defective products was 3 or 4)
C: The defective product rate was more than 20% and 30% or less (the number of defective products was 5 or 6)
D: The defective product rate was more than 30% (the number of defective products was 7 to 20)

TABLE 1

| Curable composition No. | | 101 | | 102 | | 103 | | 104 | | 105 | | 106 | |
|---|---|---|---|---|---|---|---|---|---|---|---|---|---|
| Monomer represented by | Type | A-18 | | A-1 | | A-3 | | A-6 | | A-8 | | A-9 | |
| General Formula (1) or | Formulation | 79.8 | wt % | 79.8 | wt % | 79.8 | wt % | 79.8 | wt % | 79.8 | wt % | 79.8 | wt % |
| comparative monomer | amount | | | | | | | | | | | | |
| Difunctional (meth)acrylic | Type | M-1 | | M-1 | | M-1 | | M-1 | | M-1 | | M-1 | |
| acid thioester monomer | Formulation | 19.9 | wt % | 19.9 | wt % | 19.9 | wt % | 19.9 | wt % | 19.9 | wt % | 19.9 | wt % |
| | amount | | | | | | | | | | | | |
| Photopolymerization | Formulation | 0.3 | wt % | 0.3 | wt % | 0.3 | wt % | 0.3 | wt % | 0.3 | wt % | 0.3 | wt % |
| initiator IrgTPO | amount | | | | | | | | | | | | |

| Refractive index nD of liquid of | 1.650 | 1.653 | 1.652 | 1.663 | 1.668 | 1.665 |
|---|---|---|---|---|---|---|
| composition | | | | | | |
| Refractive index nD of cured product | C (1.686) | B (1.690) | B (1.690) | A1 (1.702) | A1 (1.703) | A1 (1.702) |
| Viscosity at 25° C. | A1 (77) | A1 (70) | A1 (68) | A3 (51) | A1 (67) | A2 (58) |
| Temporal stability of liquid | A1 | A1 | A1 | A2 | A1 | A2 |
| Defective product rate of metal mold | A | A | A | A | A | A |
| release product | | | | | | |

| Curable composition No. | | 107 | | 108 | | 109 | | 110 | | 111 | | 112 | |
|---|---|---|---|---|---|---|---|---|---|---|---|---|---|
| Monomer represented by | Type | A-8 | | A-16 | | A-1/A-8 | | A-1 | | A-8 | | A-6 | |
| General Formula (1) or | Formulation | 79.8 | wt % | 79.8 | wt % | 39.9 wt %/39.9 wt % | | 99.7 | wt % | 99.7 | wt % | 99.7 | wt % |
| comparative monomer | amount | | | | | | | | | | | | |
| Difunctional (meth)acrylic | Type | M-2 | | M-1 | | M-1 | | — | | — | | — | |
| acid thioester monomer | Formulation | 19.9 | wt % | 19.9 | wt % | 19.9 | wt % | — | | — | | — | |
| | amount | | | | | | | | | | | | |
| Photopolymerization | Formulation | 0.3 | wt % | 0.3 | wt % | 0.3 | wt % | 0.3 | wt % | 0.3 | wt % | 0.3 | wt % |
| initiator IrgTPO | amount | | | | | | | | | | | | |

| Refractive index nD of liquid of | 1.658 | 1.665 | 1.661 | 1.671 | 1.689 | 1.683 |
|---|---|---|---|---|---|---|
| composition | | | | | | |
| Refractive index nD of cured product | B (1.695) | A1 (1.700) | B (1.697) | B (1.697) | A2 (1 717) | A2 (1.716) |
| Viscosity at 25° C. | A1 (75) | A1 (70) | A1 (68) | B1 (130) | B1 (132) | B2 (15) |
| Temporal stability of liquid | A1 | A1 | A1 | A1 | A1 | A2 |
| Defective product rate of metal mold | A | A | A | B | B | B |
| release product | | | | | | |

| Curable composition No. | | c11 | | c12 | | c13 | | c14 | |
|---|---|---|---|---|---|---|---|---|---|
| Monomer represented by | Type | Z-1 | | Z-2 | | Z-3 | | Z-4 | |
| General Formula (1) or | Formulation | 79.8 | wt % | 79.8 | wt % | 79.8 | wt % | 79.8 | wt % |
| comparative monomer | amount | | | | | | | | |
| Difunctional (meth)acrylic | Type | M-1 | | M-1 | | M-1 | | M-1 | |
| acid thioester monomer | Formulation | 19.9 | wt % | 19.9 | wt % | 19.9 | wt % | 19.9 | wt % |
| | amount | | | | | | | | |
| Photopolymerization | Formulation | 0.3 | wt % | 0.3 | wt % | 0.3 | wt % | 0.3 | wt % |
| initiator IrgTPO | amount | | | | | | | | |

| Refractive index nD of liquid of | 1.615 | 1.636 | 1.680 | 1.620 |
|---|---|---|---|---|
| composition | | | | |
| Refractive index nD of cured product | E (1.649) | E (1.663) | A1 (1 702) | E (1.659) |
| Viscosity at 25° C. | D (195) | D (500) | D (170) | A1 (68) |
| Temporal stability of liquid | N | N | N | N |
| Defective product rate of metal mold | C | C | D | C |
| release product | | | | |

<Notes for table>
The components in the table are as follows. The formulation amount wt % of each component described in the column of each component is % by mass, and “—” indicates that the component was not contained.

In addition, the curable composition No. 109 means that the composition contains each of A-1 and A-8, which are monomers represented by General Formula (1), at a proportion of 39.9 wt %.

### (Monomers Represented by General Formula (1))

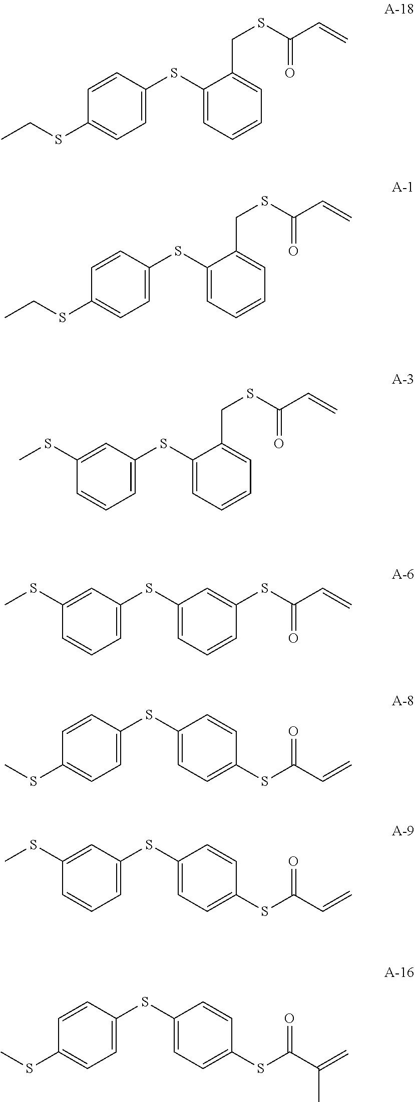

### (Comparative Monomers)

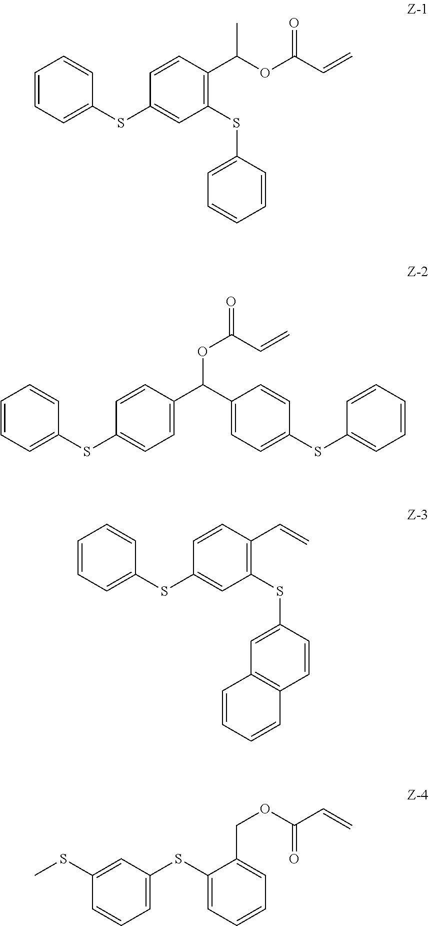

Comparative monomers Z-1 to Z-3 are compounds described in Examples 3, 8, and 10 of JP2020-37693A, respectively.

### (Difunctional (Meth)Acrylic Acid Thioester Monomers)

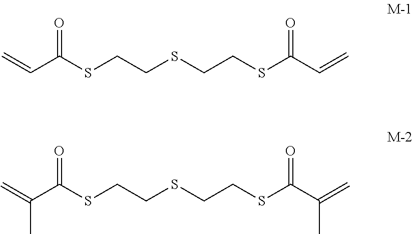

### (Photopolymerization Initiator)

IrgTPO: Irgacure TPO (trade name, manufactured by BASF Japan Ltd., available as Omnirad TPO H (trade name, manufactured by IGM Resins B.V.)), diphenyl(2,4,6-trimethylbenzoyl)phosphine oxide

From the results in Table 1, the following was found.

Both the comparative monomer Z-1 and the comparative monomer Z-2 are not the monomer represented by General Formula (1) according to the embodiment of the present invention, in that the comparative monomer Z-1 has an acryloyloxy group as a functional group and the acryloyloxy group and a benzene ring are linked by >CH(CH₃), and the comparative monomer Z-2 has an acryloyloxy group as a functional group and the acryloyloxy group and a benzene ring are linked by >CH(C₆H₄—S—C₆H₅). The curable composition Nos. c11 and c12 containing each of the comparative monomer Z-1 and Z-2 were inferior in all respects, including a high viscosity of the composition, low temporal stability of the composition, a low refractive index nD of the obtained cured product, and a high defective product rate of the metal mold release product.

The comparative monomer Z-3 is not the monomer represented by General Formula (1) according to the embodiment of the present invention in that the comparative monomer Z-3 has a vinyl group instead of a (meth)acryloylthio group as a functional group. The curable composition No. c13 containing the comparative monomer Z-3 was inferior in all respects, including a high viscosity of the composition, low temporal stability of the composition, and a high defective product rate of the metal mold release product.

The comparative monomer Z-4 is not the monomer represented by General Formula (1) according to the embodiment of the present invention in that the comparative monomer Z-4 has an acryloyloxy group instead of a (meth)acryloylthio group as a functional group. The curable composition No. c14 containing the comparative monomer Z-4 was inferior in all respects, including low temporal stability of the composition, a low refractive index nD of the obtained cured product, and a high defective product rate of the metal mold release product.

In contrast, the curable composition Nos. 101 to 109 containing the monomer represented by General Formula (1) according to the embodiment of the present invention and the difunctional (meth)acrylic acid thioester monomer were excellent in that the viscosity of the composition was as low as 77 cP or less, the temporal stability of the composition was also excellent, and in addition, the refractive index nD of the obtained cured product was as high as 1.686 or more, and the defective product rate of the metal mold release product was also suppressed to 10% or less. Above all, the curable composition Nos. 102 to 109 containing the monomer represented by General Formula (2) according to the embodiment of the present invention were more excellent in that the compositions had a low viscosity, excellent temporal stability, and excellent suppression of the defective product rate of the metal mold release product, and were also capable of further increasing the refractive index nD of the cured product. Furthermore, the curable composition Nos. 104 to 109 containing the monomer according to the embodiment of the present invention in which L in General Formula (1) is a single bond were more excellent in that the compositions had a low viscosity, excellent temporal stability, and excellent suppression of the defective product rate of the metal mold release product, and were also capable of further increasing the refractive index nD of the cured product. In particular, the curable compositions containing the monomer in which at least one of the bonding position of —S—CH₃ on the benzene ring or the bonding position of L on the benzene ring in General Formula (2) is a meta position and L is a single bond were excellent in that the compositions had a low viscosity and were capable of further improving temporal stability (Nos. 104 and 106 compared to No. 105). Above all, the curable compositions containing the monomer represented by General Formula (3) were excellent in that the viscosity of the compositions can be further reduced (No. 104 compared to No. 106).

In addition, as can be seen from the comparison between the curable composition Nos. 102 and 110, the comparison between the curable composition Nos. 105 and 111, and the comparison between the curable composition Nos. 104 and 112, the monomer represented by General Formula (1) according to the embodiment of the present invention was excellent in that, even in a case where the content proportion of the monomer (compound) represented by General Formula (1) according to the embodiment of the present invention was designed to be as high as 99.7% by mass in order to increase the refractive index nD of the cured product to 1.697 or more, the viscosity of the composition was as low as 132 cP or less, the temporal stability of the composition was also excellent, and moreover, the defective product rate of the metal mold release product was also suppressed to be 20% or less.

Although the present invention has been described in conjunction with embodiments thereof, it is not intended to limit the present invention to any of the details of the above description unless specifically stated otherwise, but it is believed that the present invention should be construed broadly without departing from the spirit and scope of the invention as set forth in the appended “WHAT IS CLAIMED IS”.

### EXPLANATION OF REFERENCES

1: curable composition
3: substrate
5: mold
7: cured product

What is claimed is:

1. A curable composition comprising:
a monofunctional (meth)acrylic acid thioester monomer represented by General Formula (1); and
a difunctional (meth)acrylic acid thioester monomer, 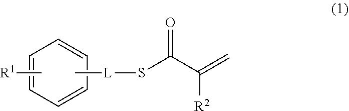
in the formula, R¹ represents a group containing an arylene group and an S atom, L represents a single bond or a methylene group, and R² represents a hydrogen atom or a methyl group.

2. The curable composition according to claim 1,
wherein a refractive index nD of a liquid at 25° C. is 1.650 or more.

3. The curable composition according to claim 1,
wherein the monofunctional (meth)acrylic acid thioester monomer represented by General Formula (1) includes a monofunctional (meth)acrylic acid thioester monomer represented by General Formula (2), 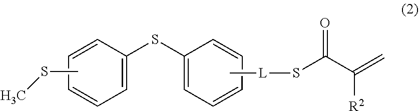
in the formula, L and R² have the same definition as L and R² described above.

4. The curable composition according to claim 1,
wherein the monofunctional (meth)acrylic acid thioester monomer represented by General Formula (1) includes a monomer in which L in General Formula (1) is a single bond.

5. The curable composition according to claim 3,
wherein the monofunctional (meth)acrylic acid thioester monomer represented by General Formula (1) includes a monomer in which at least one of a bonding position of —S—CH₃ on a benzene ring or a bonding position of L on a benzene ring in General Formula (2) is a meta position and the L is a single bond.

6. The curable composition according to claim 5,
wherein the monofunctional (meth)acrylic acid thioester monomer represented by General Formula (1) includes a monofunctional (meth)acrylic acid thioester monomer represented by General Formula (3), 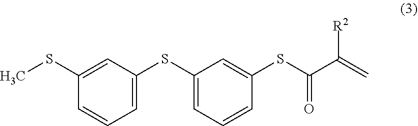
in the formula, R² has the same definition as R² described above.

7. The curable composition according to claim 1,
wherein the curable composition is for imprinting.

8. A cured product of the curable composition according to claim 1.

9. An optical material comprising:
the cured product according to claim 8.

10. A diffractive optical element which is formed of the cured product according to claim 8, comprising:
a surface having a diffraction grating shape.

11. A monofunctional (meth)acrylic acid thioester monomer represented by General Formula (1), 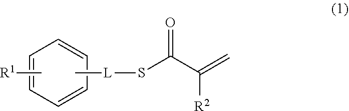
in the formula, R¹ represents a group containing an arylene group and an S atom, L represents a single bond or a methylene group, and R² represents a hydrogen atom or a methyl group.

12. A curable composition comprising:
the monofunctional (meth)acrylic acid thioester monomer represented by General Formula (1) according to claim 11.
